(19) United States
(12) Patent Application Publication
(10) Pub. No.: US 2010/0184853 A1
(43) Pub. Date: Jul. 22, 2010

(54) TERPENES WITH ANTIFUNGAL ACTIVITY AGAINST MALASSEZIA YEASTS

(75) Inventors: Librada Maria Canedo Hernandez, Armunia (Leon) (ES); Maria De Los Angeles Vinuesa-Navarro, Armunia (Leon) (ES); Antonio Fernandez-Medarde, Armunia (Leon) (ES)

Correspondence Address: HOFFMANN & BARON, LLP, 6900 JERICHO TURNPIKE, SYOSSET, NY, 11791, US

(73) Assignee: INSTITUTO BIOMAR, S.A., Armunia (Leon) (ES)

(21) Appl. No.: 12/663,766

(22) PCT Filed: Jun. 13, 2008
(86) PCT No.: PCT/EP08/57460

§ 371 Date: Mar. 8, 2010

(30) Foreign Application Priority Data
Jun. 14, 2007 (EP) 07380175.5

(51) Int. Cl. A61K 31/336; C07D 303/08; A61P 31/10; A61P 17/00; A61P 17/06

(52) U.S. Cl. 514/475; 549/559

(57) ABSTRACT

The present invention relates to the use of some terpenes, for example chlovalicin, ovalicin or fumagillin, as antifungal against Malassezia species and to their use in the manufacture of medicaments for the treatment of human skin diseases caused by these Malassezia species, such as pityriasis versicolor, seborrheic dermatitis, dandruff, atopic dermatitis, psoriasis, confluent and reticulated papillomatosis, onychomycosis, and transient acantholytic dermatosis.

### TECHNICAL FIELD

The present invention relates to the use of some terpenes as antifungal against Malassezia yeasts and to their use in the manufacture of medicaments for the treatment of human skin diseases caused by these species, such as pityriasis versicolor, seborrheic dermatitis, dandruff, atopic dermatitis, psoriasis, confluent and reticulated papillomatosis, onychomycosis, and transient acantholytic dermatosis.

### BACKGROUND OF THE INVENTION

The yeasts of the genus Malassezia (formerly known as Pityrosporum) have been associated with a number of diseases affecting the human skin, such as pityriasis versicolor, seborrheic dermatitis and dandruff, atopic dermatitis, psoriasis, and with other dermatologic less commonly disorders such as confluent and reticulated papillomatosis, onychomycosis, and transient acantholytic dermatosis (J. Am. Acad. Dermatol. 2004, 51(5), 785-98). Although Malassezia yeasts are a part of the normal microflora, under certain conditions they can cause superficial skin infection.

The commonly known therapy for these diseases is based on the use of topic preparations, such as synthetic detergents (for instance, lotions and shampoos), containing antimycotics such as for instance climbazol, ketoconazol, poctone-olamine, zinc pyrithione, selenium sulphide or other sulphur-containing substances. These topic antifungal preparations are often combined with a cortisonic drug to control the inflammation and alleviate the pain and itching. However, the use of these molecules do not result fully satisfactory, and in some cases these molecules exhibit in fact an intrinsic toxicity. These types of treatment are moreover always associated with a high incidence of recurrence of skin surface mycoses. For instance, the relapse rate of acute mycosis is estimated at 20% of the population affected by pityriasis versicolor and treated with ketoconazol within 6 months from the date of suspending the therapy, and at 50% after one year. Based on these findings, there is a need for compounds which demonstrate antifungal activity against Malassezia yeasts.

The problem to be solved by the present invention is to provide compounds that are useful in the treatment of diseases caused by yeasts of the genus Malassezia.

Fumagillin, a product from Aspergillus fumigatus, was used in the 1950s to treat human amebiasis and is still being used to treat honeybees with Nosema apis microsporidial infections (Science, 1952, 115, 70-71 and 71-72). Fumagillin also inhibited replication of E. cuniculi in tissue culture (J. Protozool. 1980, 27, 202-208) and has been applied topically to treat ocular microsporidial infections (Expert Rev. Anti-Infect. Ther. 2005, 3, 419-434), and it is effective against intestinal microsporidiosis and Cryptococcus (Antimicrob. Agents Chemother. 2006, 50, 2146-2155). Fumagillin and synthetic analogues have been disclosed to inhibit angiogenesis and suppress tumour growth (Nature, 1990, 348, 555-557).

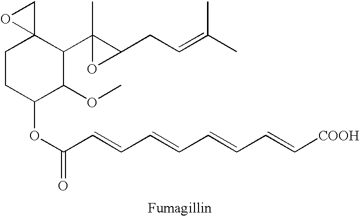

Ovalicin, a product from Pseudorotium ovalis (Hely. Chim. Acta, 1973, 56, 819-830; Hely. Chim. Acta, 1968, 51, 1395-1408), has been disclosed with antimicrosporidia (Antimicrob. Agents Chemother. 2006, 50, 2146-2155), antiangiogenic (Protein Sci. 2006, 15, 1842-8), and immunosuppressant (Eur. J. Biochem. 1981, 118, 143-50) activies.

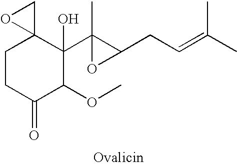

Chlovalicin, a product from Pseudallesheria sp, has been disclosed with cytotoxic (J. Antibiot. 1996, 49, 631-4) and immunosuppressant (JP7017957) activities.

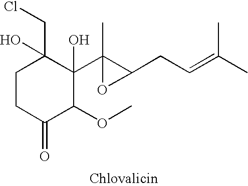

FR-65814 (JP61033181) with immunosuppressant activity, FR-111142 and fumagillol (JP2233610, J. Antibiotic. 1992, 45, 348-54), cis-fumagillin (J. Antibiot. 2000, 53, 799-806), and FR-125035 (JP304712) all of them with antiangiogenic activity, are some other examples of disclosures of this type of terpenes isolated from different microorganisms. There are a huge number of synthetic derivatives of this type of terpenes, mainly focused as angiogenesis inhibitors and with some of them in different clinical trials, for example PPI-2458 (Methods Find. Exp. Clin. Pharmacol. 2007, 29, 153-73) or CKD-732 (Biol. Pharm. Bull. 2005, 28, 217-23).

The antifungal activity against Malassezia yeasts for these known terpenes has not been previously disclosed.

### SUMMARY OF THE INVENTION

The present invention is directed to the use of terpenes of general formula (Ia) or (Ib) or (Ic) as antifungal against Malassezia yeasts and to their use in the manufacture of medicaments for the treatment of human skin diseases caused by these species,

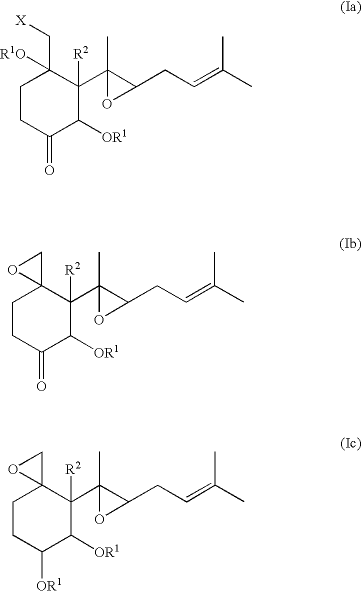

wherein the R¹ groups are each independently selected from the group consisting of hydrogen, acyl, alkyl, alkenyl, alkynyl, aryl, —CONH₂, alkali metal, and sugar, and wherein R² is selected from hydrogen and OR¹.

In another aspect, the present invention is also directed to the use of pharmaceutically acceptable salts, solvates, derivatives or stereoisomers of compounds of formula (Ia) or (Ib) or (Ic) in the treatment of diseases caused by Malassezia species, or in the preparation of a medicament for the treatment of diseases caused by Malassezia species.

### DETAILED DESCRIPTION OF THE INVENTION

The present invention is directed to the use of terpenes of general formula (Ia) or (Ib) or (Ic):

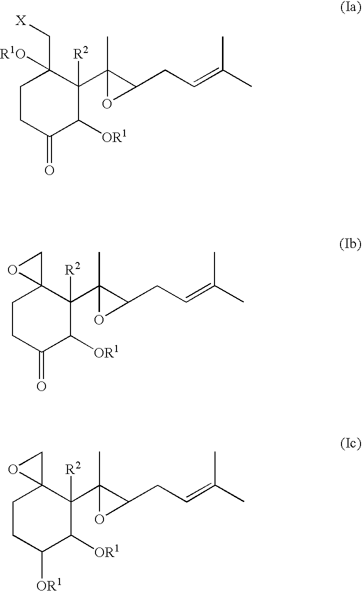

wherein the R¹ groups are each independently selected from the group consisting of hydrogen, acyl, alkyl, alkenyl, alkynyl, aryl, —CONH₂, alkali metal, and sugar, and wherein R² is selected from hydrogen and OR¹,
as antifungal against Malassezia yeasts and to their use in the manufacture of medicaments for the treatment of human skin diseases caused by these species, such as pityriasis versicolor, seborrheic dermatitis, dandruff, atopic dermatitis, psoriasis, confluent and reticulated papillomatosis, onychomycosis, and transient acantholytic dermatosis,

In another aspect, the present invention is also directed to the use of pharmaceutically acceptable salts, solvates, derivatives or stereoisomers of compounds of formula (Ia) or (Ib) or (Ic) in the treatment of diseases caused by Malassezia species, or in the preparation of a medicament for the treatment of diseases caused by Malassezia species.

In the above definition of compounds of formula (Ia) or (Ib) or (Ic) the following terms have the meaning indicated:

An acyl group is of the form R³CO—, wherein R³ is an organic group. Suitable acyl groups have from 2 to about 12 carbon atoms, more preferably from 2 to about 8 carbon atoms, still more preferably from 2 to about 6 carbon atoms, even more preferably 2 carbon atoms.

Alkyl group preferably have from 1 to 20 carbon atoms. As used herein, the term alkyl, unless otherwise modified, refers to both cyclic and non-cyclic groups, although cyclic groups will comprise at least three carbon ring members. Non-cyclic alkyl refers to a straight-chain or branched alkyl group.

Preferred alkenyl and alkynyl groups in the compounds of the present invention have one or more unsaturated linkages and from 2 to about 20 carbon atoms. The terms alkenyl and alkynyl as used herein refer to both cyclic and non cyclic groups. Non-cyclic alkenyl or alkynyl refers to a straight-chain or branched alkenyl or alkynyl group.

The groups above mentioned may be substituted at one or more available positions by one or more suitable groups such as OR′, ═O, SR′, SOR′, SO₂R′, NO₂, NHR′, N(R′)₂, ═N—R′, NHCOR′, N(COR′)₂, NHSO₂R′, CN, halogen, C(═O)R′, CO₂R′, OC(═O)R′ wherein each of the R′ groups is independently selected from the group consisting of H, OH, NO₂, NH₂, SH, CN, halogen, ═O, C(═O)H, C(═O)CH₃, CO₂H, substituted or unsubstituted C1-C12 alkyl, substituted or unsubstituted C2-C12 alkenyl, substituted or unsubstituted C2-C12 alkynyl and substituted or unsubstituted aryl.

“Aryl” refers to single and multiple ring radicals, including multiple ring radicals that contain separate and/or fused aryl groups. Typical aryl groups contain from 1 to 3 separated or fused rings and from 6 to about 18 carbon ring atoms, such as phenyl, naphthyl, indenyl, fenanthryl or anthracyl radical.

The aryl group in the compounds of the present invention may be substituted at one or more available positions by one or more suitable groups, e. g., halogen such as F, Cl, Br and I.; cyano; hydroxyl; nitro; azido; alkanoyl such as a C1-6 alkanoyl group such as acyl and the like; carboxamido; alkyl groups including those groups having 1 to about 12 carbon atoms or from 1 to about 6 carbon atoms and more preferably 1-3 carbon atoms; alkenyl and alkynyl groups including groups having one or more unsaturated linkages and from 2 to about 12 carbon or from 2 to about 6 carbon atoms; alkoxy groups having one or more oxygen linkages and from 1 to about 12 carbon atoms or 1 to about 6 carbon atoms; aryloxy such as phenoxy; alkylthio groups including those moieties having one or more thioether linkages and from 1 to about 12 carbon atoms or from 1 to about 6 carbon atoms; alkylsulfinyl groups including those moieties having one or more sulfinyl linkages and from 1 to about 12 carbon atoms or from 1 to about 6 carbon atoms ; alkylsulfonyl groups including those moieties having one or more sulfonyl linkages and from 1 to about 12 carbon atoms or from 1 to about 6 carbon atoms; aminoalkyl groups such as groups having one or more N atoms and from 1 to about 12 carbon atoms or from 1 to about 6 carbon atoms; aralkyl such as benzyl. Unless otherwise indicated, an optionally substituted group may have a substituent at each substitutable position of the group, and each substitution is independent of the other.

Notable alkali metals include sodium or potassium.

“Sugar” refers to mono-, di- or tri-saccharides or saccharide derivatives, preferably mono- or di-saccharides. Pentose or hexose compounds are preferred. Derivatives include sugar glycosides, N-glycosylamines, O-acyl derivatives, O-methyl derivatives, sugar alcohols, sugar acids, and deoxy sugars.

The term “pharmaceutically acceptable salts, derivatives” refers to any pharmaceutically acceptable salt, ester, solvate, hydrate or any other compound which, upon administration to the recipient is capable of providing (directly or indirectly) a compound as described herein. The preparation of salts and derivatives can be carried out by methods known in the art.

For instance, pharmaceutically acceptable salts of compounds provided herein are synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

The compounds of the invention may be in crystalline form either as free compounds or as solvates (e.g. hydrates) and it is intended that both forms are within the scope of the present invention. Methods of solvation are generally known within the art.

The compounds of the present invention represented by the above described formula (Ia) or (Ib) or (Ic) may include enantiomers depending on their asymmetry or diastereoisomers. The single isomers and mixtures of the isomers fall within the scope of the present invention.

Pharmaceutical compositions comprise a compound of formula (Ia) or (Ib) or (Ic) or a pharmaceutically acceptable salt, solvate, derivative, or stereoisomer thereof together with a pharmaceutically acceptable carrier, adjuvant, or vehicle, for administration to a patient.

Examples of pharmaceutical compositions include any solid (tablets, pills, capsules, granules etc.) or liquid (solutions, suspensions or emulsions) composition for oral, topical or parenteral administration.

Administration of the compounds of formula (Ia) or (Ib) or (Ic) or compositions thereof may be by any suitable method, such as oral preparations and topical administration. In a preferred embodiment the pharmaceutical compositions are in topical form.

Pharmaceutical compositions of the invention, suitable for topical administration are for example creams, lotions, ointments, microemulsions, fatty ointments, gels, emulsion-gels, pastes, foams, tinctures, solutions, patches, bandages and transdermal therapeutic systems. Most preferred are creams or emulsion-gels.

Creams or lotions are oil-in-water emulsions. Oily bases that can be used are fatty alcohols, especially those containing from 12 to 18 carbon atoms, for example lauryl, cetyl or stearyl alcohol, fatty acids, especially those containing from 10 to 18 carbon atoms, for example palmitic or stearic acid, fatty acid esters, e.g. glyceryl tricaprilocaprate (neutral oil) or cetyl palmitate, liquid to solid waxes, for example isopropyl myristate, wool wax or beeswax, and/or hydrocarbons, especially liquid, semi-solid or solid substances or mixtures thereof, for example petroleum jelly (petrolatum, Vaseline) or paraffin oil. Suitable emulsifiers are surface-active substances having predominantly hydrophilic properties, such as corresponding non-ionic emulsifiers, for example fatty acid esters of polyalcohols and/or ethylene oxide adducts thereof, especially corresponding fatty acid esters with (poly)ethylene glycol, (poly)propylene glycol or sorbitol, the fatty acid moiety containing especially from 10 to 18 carbon atoms, especially partial glycerol fatty acid esters or partial fatty acid esters of polyhydroxyethylene sorbitan, such as polyglycerol fatty acid esters or polyoxyethylene sorbitan fatty acid esters (Tweens), and also polyoxyethylene fatty alcohol ethers or fatty acid esters, the fatty alcohol moiety containing especially from 12 to 18 carbon atoms and the fatty acid moiety especially from 10 to 18 carbon atoms, such as polyhydroxyethyleneglycerol fatty acid ester (for example Tagat S), or corresponding ionic emulsifiers, such as alkali metal salts of fatty alcohol sulfates, especially having from 12 to 18 carbon atoms in the fatty alcohol moiety, for example sodium lauryl sulfate, sodium cetyl sulfate or sodium stearyl sulfate, which are usually used in the presence of fatty alcohols, for example cetyl alcohol or stearyl alcohol. Additives to the aqueous phase are, inter alia agents that prevent the creams from drying out, for example humectants, such as polyalcohols, such as glycerol, sorbitol, propylene glycol and/or polyethylene glycols, and also preservatives, perfumes, gelling agents, etc.

Ointments are water-in-oil emulsions that contain up to 70%, but preferably from approximately 20% to approximately 50%, water or aqueous phase. Suitable as fatty phase are especially hydrocarbons, for example petroleum jelly, paraffin oil and/or hard paraffins, which, in order to improve the water-binding capacity, preferably contain suitable hydroxy compounds, such as fatty alcohols or esters thereof, for example cetyl alcohol or wool wax alcohols, or wool wax or beeswax. Emulsifiers are corresponding lipophilic substances, for example of the type indicated above, such as sorbitan fatty acid esters (Spans), for example sorbitan oleate and/or sorbitan isostearate. Additives to the aqueous phase are, inter alia humectants, such as polyalcohols, for example glycerol, propylene glycol, sorbitol and/or polyethylene glycol, and also preservatives, perfumes, etc.

Microemulsions are isotropic systems based on the following four components: water, a surfactant, for example a tensioactive, a lipid, such as a non-polar or polar oil, for example paraffin oil, natural oils such as olive or maize oil, and an alcohol or polyalcohol containing lipophilic groups, for example 2-octyldodecanol or ethoxalated glycerol or polyglycerol esters. If desired, other additives may be added to the microemulsions. Microemulsions have micelles or particlaes with sizes below 200 nm and are transparent or translucid systems, the form spontaneoulsy and are stable.

Fatty ointments are water-free and contain as base especially hydrocarbons, for example paraffin, petroleum jelly and/or liquid paraffins, also natural or partially synthetic fat, such as fatty acid esters of glycerol, for example coconut fatty acid triglyceride, or preferably hardened oils, for example hydrogenated groundnut oil, castor oil or waxes, also fatty acid partial esters of glycerol, for example glycerol mono- and di-stearate, and also, for example, the fatty alcohols increasing the water-absorption capacity, emulsifiers and/or additives mentioned in connection with the ointments.

With gels, a distinction is made between aqueous gels, water-free gels and gels having a low water content, which gels consist of swellable, gel-forming materials. There are used especially transparent hydrogels based on inorganic or organic macromolecules. High molecular weight inorganic components having gel-forming properties are predominantly water-containing silicates, such as aluminium silicates, for example bentonite, magnesium aluminium silicates, for example Veegum, or colloidal silicic acid, for example Aerosil. As high molecular weight organic substances there are used, for example, natural, semisynthetic or synthetic macromolecules. Natural and semi-synthetic polymers are derived, for example, from polysaccharides containing a great variety of carbohydrate components, such as celluloses, starches, tragacanth, gum arabic and agar-agar, and gelatin, alginic acid and salts thereof, for example sodium alginate, and derivatives thereof, such as lower alkylcelluloses, for example methyl- or ethyl-cellulose, carboxy- or hydroxy-lower alkylcelluloses, for example carboxymethyl- or hydroxyethyl-cellulose. The components of synthetic gel-forming macromolecules are, for example, suitably substituted unsaturated aliphatic compounds such as vinyl alcohol, vinylpyrrolidine, acrylic or methacrylic acid.

Emulsion-gels—also called “emulgels”—represent topical compositions which combine the properties of a gel with those of an oil-in-water emulsion. In contrast to gels, they contain a lipid phase which due to its fat-restoring properties enables the formulation to be massaged in whilst, at the same time, the direct absorption into the skin is experienced as a pleasant property. Furthermore, one can observe an increased solubility for lipophilic active ingredients. One advantage of emulsion-gels over oil-in-water emulsions resides in the enhanced cooling effect which is brought about by the coldness due to evaporation of the additional alcohol component, if present.

Foams are administered, for example, from pressurised containers and are liquid oil-in water emulsions in aerosol form; unsubstituted hydrocarbons, such as alkanes, for example propane and/or butane, are used as propellant. As oil phase there are used, inter alia hydrocarbons, for example paraffin oil, fatty alcohols, for example cetyl alcohol, fatty acid esters, for example isopropyl myristate, and/or other waxes. As emulsifiers there are used, inter alia, mixtures of emulsifiers, having predominantly hydrophilic properties, such as polyoxyethylene sorbitan fatty acid esters (Tweens), and emulsifiers having predominantly lipophilic properties, such as sorbitan fatty acid esters (Spans). The customary additives, such as preservatives, etc., are also added.

Tinctures and solutions generally have an ethanolic base, to which water may be added and to which there are added, inter alia, polyalcohols, for example glycerol, glycols and/or polyethylene glycol, as humectants for reducing evaporation, and fat-restoring substances, such as fatty acid esters with low molecular weight polyethylene glycols, propylene glycol or glycerol, that is to say lipophilic substances that are soluble in the aqueous mixture, as a replacement for the fatty substances removed from the skin by the ethanol, and, if necessary, other adjuncts and additives. Suitable tinctures or solutions may also be applied in spray form by means of suitable devices.

Transdermal therapeutic systems contain an effective amount of the compounds of the invention optionally together with a carrier. Useful carriers comprise absorbable pharmacological suitable solvents to assist passage of the active ingredient through the skin. Transdermal delivery systems are, for example, in the form of a patch comprising (a) a substrate (=backing layer or film), (b) a matrix containing the active ingredient, optionally carriers and optionally (but preferably) a special adhesive for attaching the system to the skin, and normally (c) a protection foil (=release liner). The matrix (b) is normally present as a mixture of all components or may consist of separate layers.

All these systems are well known to the person skilled in the art. The manufacture of the topically administrable pharmaceutical preparations is effected in a manner known per se, for example by dissolving or suspending the compounds as defined above in the base or, if necessary, in a portion thereof.

The compositions according to the invention may also comprise conventional additives and adjuvants for dermatological applications, such as preservatives, especially paraben esters like methylparaben, ethylparaben, propylparaben, butylparaben, or quaternary ammonium compounds like benzalkonium chloride, or formaldehyde donors like imidazonidinyl urea, or alcohols like benzyl alcohol, phenoxyethanol or acids like benzoic acid, sorbic acid; acids or bases used as pH buffer excipients; antioxidants, especially phenolic antioxidants like hydroquinone, tocopherol and derivatives thereof, as well as flavonoids, or miscellaneous antioxidants like ascorbic acid,ascorbyl palmitat ; perfumes; fillers such as kaolin or starch; pigments or colorants ; UV-screening agents; moisturizers, especially glycerin, butylen glycol, hexylen glycol, urea, hyaluronic acid or derivatives thereof; anti-free radical agents such as vitamin E or derivatives thereof; penetration enhancers especially propylene glycol; ethanol; isopropanol; dimethylsulfoxide; N-methyl-2-pyrrolidone; fatty acids/alcohols such as oleic acid, oleyl alcohol; terpenes such as limonen, menthol, 1-8 cineole; alkyl esters such as ethyl acetate, butyl acetate; ion pairing agents such as salicylic acid.

Further details concerning suitable topical formulations may be obtained by reference to standard textbooks such as Banker and Rhodes (Ed) Modern Pharmaceutics 4^{th} ed. (2002) published by Marcel Dekker Inc.; Harry's Cosmeticology (2000), 8th Edition, Chemical Publishing Co.; Remington's Pharmaceutical Sciences 20^{th} ed Mack Publishing Co. (2000).

The compounds of formula (Ia) or (Ib) or (Ic) and compositions thereof may be used with other drugs to provide a combination therapy. The other drugs may form part of the same composition, or be provided as a separate composition for administration at the same time or at different time.

Particularly preferred compounds falling under the general formulas (Ia) or (Ib) or (Ic) are the known compounds chlovalicin, ovalicin, and fumagillin.

Chlovalicin, ovalicin, and fumagillin are preferably obtained by isolation from microorganisms as was previously described. In a similar way, any compound of formulas (Ia) or (Ib) or (Ic) can be obtained with known organic synthetic procedures (Angew. Chem. Int. Ed. Engl. 2006, 45, 789-93; J. Org. Chem. 2005, 70, 10162-5; Org. Biomol. Chem. 2005, 3, 2150-4; J. Am. Chem. Soc. 1994, 116, 12109-12110).

### Examples

Fungal Culture

Antifungal activity was tested against Malassezia furfur CBS 1878.

Malassezia furfur was grown on ML2 agar plates containing potato dextrose agar 4.2%, ox bile 0.8%, glycerol 1% (v:v), olive oil 1% (v:v), tween 60 0.5% (v:v) for two days, at 30° C., as stock culture.

Antifungal Disk Diffusion Susceptibility Test

An area of 2 cm² of a growing plate of Malassezia furfur stock culture was scraped using a sterile loop. Biomass was homogenated in 1 ml glycerol 20%. A volume of 100 μl of the glycerol homogenate was spread on testing ML2 plates.

Each test compound was dissolved in distilled water and added on blank paper discs (6 mm, diameter) on the inoculated plates. Plates were incubated for two days at 30° C. Inhibition circles around the discs (halo) were used as activity measure.

Ketoconazol was used as inhibition control.

The test was carried out at 15 nmol for each compound. Antifungal activities are shown at table 1.

Table 1 illustrates the antifungal activity data against Malassezia furfur for the known compounds chlovalicin, ovalicin, fumagillin, and ketoconazol

| | Halo (mm) |
|---|---|
| Chlovalicin | 35 |
| Ovalicin | 46 |
| Fumagillin | 27 |
| Ketoconazol | 23 |

1. Use of a compound of formula (I) 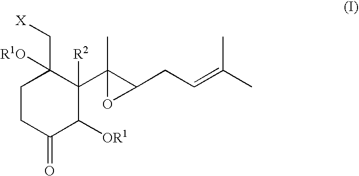
wherein the R¹ groups are each independently selected from the group consisting of hydrogen, acyl, alkyl, alkenyl, alkynyl, aryl, —CONH₂, alkali metal, and sugar, and wherein R² is selected from hydrogen and OR¹, or pharmaceutically acceptable salts, solvates, derivatives or stereoisomers thereof, in the preparation of a medicament for the treatment of human skin diseases caused by Malassezia species.

2. Use according to claim 1, wherein the disease is pityriasis versicolor, seborrheic dermatitis, dandruff, atopic dermatitis, psoriasis, confluent and reticulated papillomatosis, onychomycosis, and transient acantholytic dermatosis.

3. Use according to claim 1, wherein the Malassezia specie is Malassezia furfur.

4. Use according to claims 1, wherein the compound of formula (I) is chlovalicin.

5-6. (canceled)

7. A method of treating human skin diseases caused by Malassezia species with a compound of formula (I) as defined in claim 1, which method comprises administering to a patient in need of such a treatment a therapeutically effective amount of a compound of formula (I), or pharmaceutically acceptable salts, solvates, derivatives or steroisomers thereof, or a pharmaceutical composition thereof.
